(19) United States
(12) Patent Application Publication
(10) Pub. No.: US 2025/0241328 A1
(43) Pub. Date: Jul. 31, 2025

(54) REDUCTANT TREATMENT METHODS FOR FOOD PRODUCT PRECURSORS

(71) Applicant: Impossible Foods Inc., Redwood City, CA (US)

(72) Inventors: Innu Chaudhary, Redwood City, CA (US); Rachel Fraser, San Francisco, CA (US); Allen Henderson, Brisbane, CA (US); Chun-Ta Huang, Fremont, CA (US); Anthony Mauriello, Newark, CA (US); Dunilka Ratnayaka, San Jose, CA (US); Chi Heng Wu, Santa Clara, CA (US)

(21) Appl. No.: 18/855,779

(22) PCT Filed: Apr. 11, 2023
(86) PCT No.: PCT/US2023/018176

§ 371 Date: Oct. 10, 2024

Related U.S. Application Data
(60) Provisional application No. 63/329,819, filed on Apr. 11, 2022.

(51) Int. Cl. A23B 2/767; A23B 2/40; A23B 2/771; A23B 2/788

(52) U.S. Cl. CPC A23B 2/767; A23B 2/40; A23B 2/771; A23B 2/788

(57) ABSTRACT

Reductants may reduce microbial bioburden in protein solutions. Reducing bioburden can increase shelf life or ensure the safety of food or pharmaceutical products. The use of reductants to reduce bioburden in producing non-animal-based food products may also have additional advantages. For example, chroma and hue angle may be improved so that the non-animal-based food products may closely resemble the appearance of animal-based food products. Embodiments may include a method for forming a food product precursor. The method may include providing a first mixture. The first mixture may include a protein. The method may further include adding a reductant to the first mixture to form a second mixture. The method may also include heating the second mixture at a temperature for a duration to form a third mixture. The third mixture may be the food product precursor. Embodiments may include the food product precursor.

This application is a nonprovisional of and claims the benefit of priority to U.S. Provisional Patent Application No. 63/329,819, filed Apr. 11, 2022, the entire contents of which is incorporated herein by reference for all purposes.

### TECHNICAL FIELD

The technical field involves methods for forming food product precursors, food product precursors, methods for reducing bioburden in an aqueous composition, proteins for food (e.g., food ingredient, flavoring agent, coloring agent, gelling agent, binding agent, nutritional supplement), proteins for pharmaceuticals (e.g., therapeutic, biologic, vaccines), and sterile proteins.

### BACKGROUND

As standards of living have improved around the world, there has been a corresponding increase in demand for animal-based food products such as beef, pork, lamb, goat, poultry, fish, and shellfish, among other categories of animal-based foods. Unfortunately, this increase in demand for animal-based foods has created many environmental and ethical challenges. The challenges include increased pollution from animal waste and greenhouse gases, more annexation of farmland and green spaces to raise animals, overfishing of lakes and oceans, and overcrowded conditions for the animals, among others. These challenges have not reduced consumer demand, and further increased production of animal-based meat products seems inevitable without appealing non-animal-based substitutes.

Thus, there is a need for better non-animal-based food products that more closely simulate the experience of preparing and eating animal-based meat and other food products. These and other needs are addressed by the present technology.

### SUMMARY

Reductants may reduce microbial bioburden in protein solutions. Reducing bioburden can increase shelf life or ensure the safety of food or pharmaceutical products. The use of reductants to reduce bioburden in producing non-animal-based food products may also have additional advantages. For example, chroma and hue angle may be improved so that the non-animal-based food products may closely resemble the appearance of animal-based food products.

Embodiments may include a method for forming a food product precursor. The method may include providing a first mixture. The first mixture may include a protein. The method may further include adding a reductant to the first mixture to form a second mixture. The method may also include heating the second mixture at a temperature for a duration to form a third mixture. The third mixture may be the food product precursor.

Embodiments may include a food product precursor. The food product precursor may include a heme-containing protein. The food product precursor may also include a cysteine having a first concentration in a range of 5 mM to 50 mM. The food product precursor may have a pH of 5.5 or higher.

Embodiments may include a method of reducing bioburden in a first mixture. The method may include providing the first mixture comprising the protein. The method may also include adding a reductant to the first mixture to form a second mixture. The method may further include heating the second mixture at a temperature for a duration to form a third mixture. The third mixture may have a lower bioburden than the first mixture.

### BRIEF DESCRIPTION OF THE DRAWINGS

A further understanding of the nature and advantages of the disclosed technology may be realized by reference to the remaining portions of the specification and the drawings.

FIGS. 1A and 1B show aerobic plate count and lactic acid bacteria of HTST-treated heme-containing protein according to embodiments of the present invention.

FIGS. 2A, 2B, and 2C are tables showing the reduction of pathogens in leghemoglobin samples with and without cysteine according to embodiments of the present invention.

FIG. 3 shows LegH titer loss through HTST according to embodiments of the present invention.

FIG. 4 shows percent suspended solids results according to embodiments of the present invention.

FIG. 5 shows pressure versus time for HTST treatment according to embodiments of the present invention.

FIG. 6 shows chroma and hue angle results before and after HTST with cysteine for a leghemoglobin solution according to embodiments of the present invention.

FIGS. 7A and 7B show color protection of LegH Prep under different conditions according to embodiments of the present invention.

FIG. 8 shows images of capillary tubes used to test the reduction of the testing organisms according to embodiments of the present invention.

FIG. 9 shows thermal inactivation of Salmonella cocktail according to embodiments of the present invention.

FIG. 10 shows D value estimates versus temperature for Salmonella cocktail according to embodiments of the present invention.

FIG. 11 shows thermal inactivation of L. monocytogenes cocktail according to embodiments of the present invention.

FIG. 12 shows D value estimates versus temperature for L. monocytogenes cocktail according to embodiments of the present invention.

FIG. 13 shows thermal inactivation of E. coli O157 cocktail according to embodiments of the present invention.

FIG. 14 shows D value estimates versus temperature for E. coli O157 cocktail according to embodiments of the present invention.

FIG. 15 is a flowchart of a process of forming a food product precursor according to embodiments of the present invention.

### DETAILED DESCRIPTION

Embodiments described herein include methods for forming food product precursors, food product precursors, methods for reducing bioburden in an aqueous composition, proteins for food (e.g., food ingredient, flavoring agent, coloring agent, gelling agent, binding agent, nutritional supplement), proteins for pharmaceuticals (e.g., therapeutic, biologic, vaccines), sterile proteins, and methods for reducing bioburden of a mixture of such proteins. A food product precursor may be a component of a food product or one or more processing steps away from a food product. For example, the food product precursor may undergo one or more of concentrating, purifying, drying, heating, cooking (e.g., from the simulated appearance of raw animal meat to cooked animal meat), drying, or flavoring before becoming a food product. The food product may be suitable for human consumption and/or may be the final product to be sold to a consumer. The food product may be a product that is intended to be cooked before consumed.

### I. Increase Microbial Log Reduction

The application of use of reductants, alone or in combination with other treatments, can reduce microbial bioburden in liquid products, including protein solutions. We show that adding a food-safe reductant, such as L-cysteine, reduces bioburden (CFU/mL) (colony forming units per milliliter) in a liquid food ingredient. Using a reductant can be combined with thermal processing to reduce bioburden. pH can also modulate the activity (i.e., higher pH can increase the antimicrobial action of the reductant).

The addition of 15 mM and 35 mM cysteine to heme samples at pH 9.5 decreases bacterial bioburden CFU/g (colony forming units per gram) following high temperature short time (HTST) treatment.

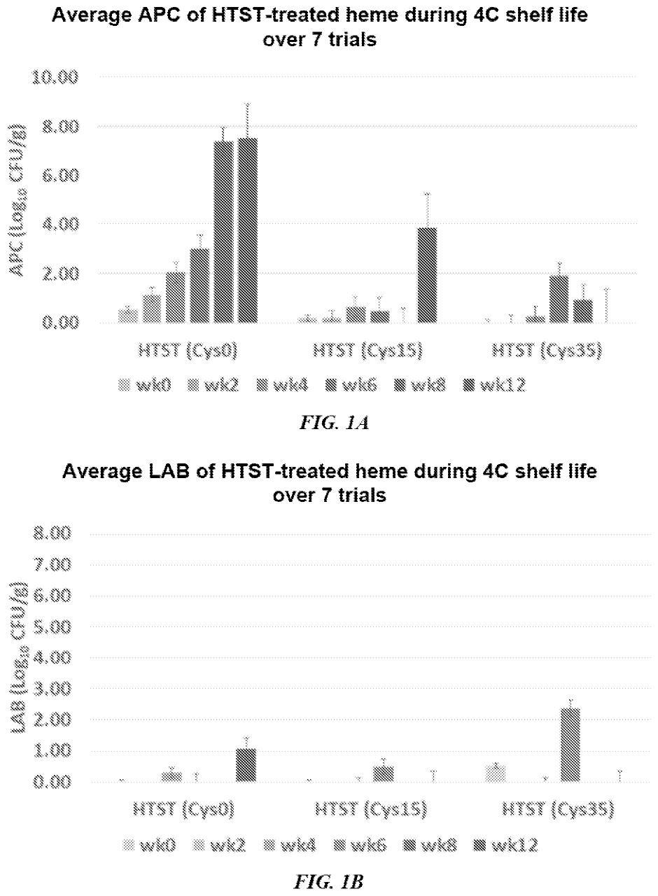

Refrigerated shelf-life studies of an HTST-treated heme-containing protein, soy leghemoglobin (LegH), demonstrated a cysteine-dependent increase in aerobic plate count (APC) stability (FIG. 1A). APC captures both gram negative and gram positive bacteria, whereas lactic acid bacteria (LAB) captures gram positive bacteria (FIG. 1B).

Cysteine treatment during HTST significantly increased the CFU/g reduction of pathogens Salmonella, E. coli, and Listeria. Salmonella and E. coli are gram negative bacteria, and Listeria is gram positive. Addition of cysteine reduced the CFU/g of E. coli even in the absence of heat.

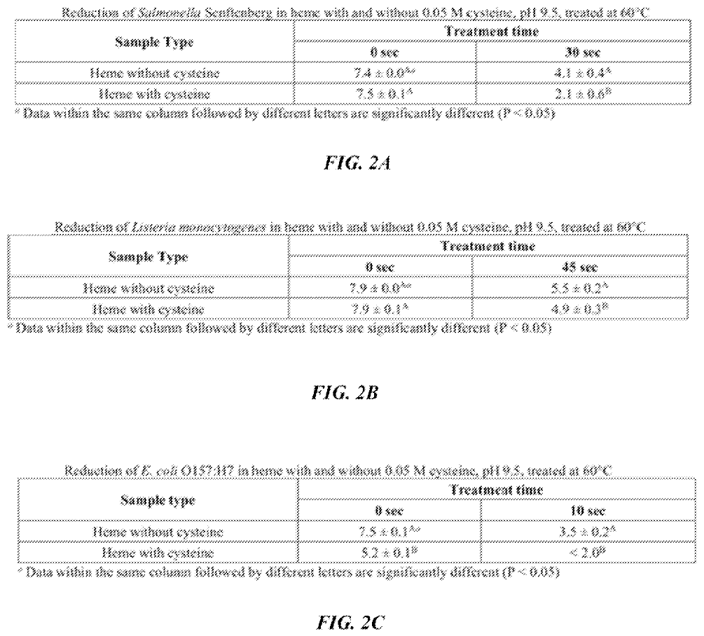

FIGS. 2A, 2B, and 2C are tables showing the reduction of pathogens in leghemoglobin samples with and without cysteine.

Food safe reductants are a novel, safe, and cheap method to control bioburden in liquid products (e.g., in liquid ingredients). They have the further advantage of creating savory flavors in flavor systems, meaning that the additive can support both bioburden reduction and flavor generation.

Use of cysteine in HTST may enable pathogen reduction at lower temperatures and/or residence times. This could be beneficial for temperature-sensitive products such as proteins, including heme-containing proteins. Proteins can denature or aggregate at high temperatures, and some metalloproteins such as heme-containing proteins can oxidize at higher temperatures. Increased log reduction of microbes such as gram-negative bacteria can increase product shelf life.

### II. Increasing Protein Thermostability

Reductants can make proteins more soluble by preventing intermolecular disulfide bonds from forming larger protein complexes and suspended solids which can participate in aggregation. By stabilizing proteins, reductants can decrease protein aggregation.

Reductants like cysteine can also stabilize metal cofactors that are susceptible to oxidation. For example, a reducing environment can stabilize oxygen-bound LegH by keeping the heme iron in the +2, rather than +3, oxidation state, which is required to bind oxygen. Increased temperature or reduced pH may accelerate heme oxidation.

The addition of cysteine into a protein solution of soy leghemoglobin protein recombinantly produced in Pichia (LegH Prep) prior to high temperature short time (HTST) thermal treatment resulted in increased protein thermostability.

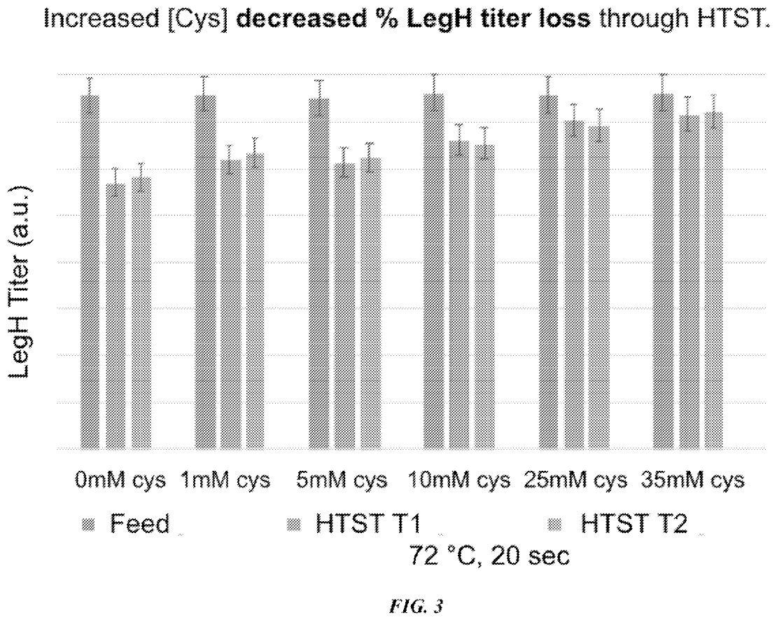

HTST treatment of LegH Prep for 20 seconds at elevated temperatures (e.g., 68-72° C.) resulted in a decrease in LegH concentration (mg/g) as measured by ultra-performance liquid chromatography (UPLC). This decrease in concentration is due to LegH aggregating or denaturing in response to heat. In the presence of cysteine, the decrease in LegH concentration was diminished in a dose-dependent manner, suggesting that cysteine increases the thermostability of LegH. FIG. 3 shows LegH titer results. For UPLC, there is a resolved peak at the LegH retention time that can be tracked by 415 nm absorbance but the integration is performed on the 280 nm peak at that position.

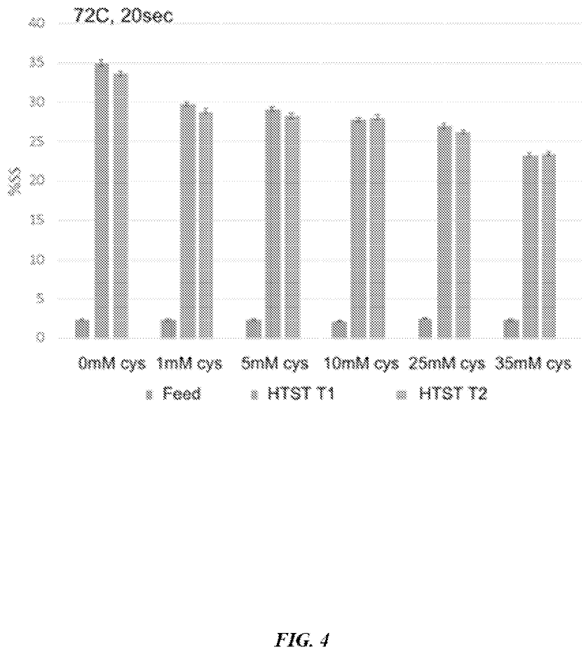

HTST treatment of LegH Prep for 20 seconds at temperatures ranging from 68-72° C. resulted in an increase in percent suspended solids (% SS). Suspended solid formation is likely due to aggregation or denaturation of the Pichia proteins within LegH Prep in response to heat. In the presence of cysteine, the increase in % SS was diminished in a dose-dependent manner, suggesting that cysteine may stabilize Pichia proteins. FIG. 4 shows percent suspended solids results. % SS is a weight-based measurement of the remaining wet pellet following centrifugation of the sample. Increased suspended solids changes the physical properties of the protein solution and are typically associated with increased viscosity.

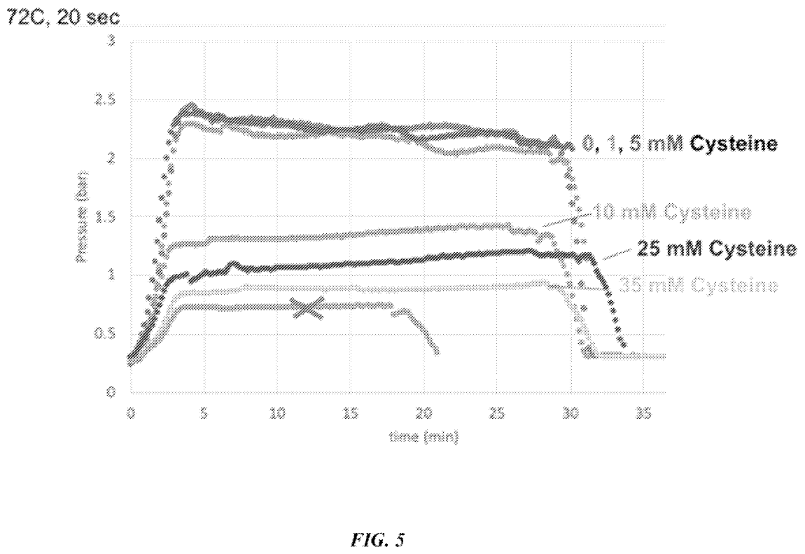

HTST treatment of LegH Prep for 20 seconds at temperatures ranging from 68-72° C. results in an increase in pressure within the HTST equipment. This increased pressure is correlated with viscosity increases and due to the thickening of protein solutions caused by denaturation or gelation due to heat. This will eventually cause the formation of a protein gel layer inside the piping, often referred to as “burn on”. Burn on can eventually lead to equipment failure and diminished product quality. In the presence of cysteine, this pressure increase was diminished in a dose-dependent manner. The reduced suspended solids described above can contribute to this decrease in pressure. FIG. 5 shows pressure versus time.

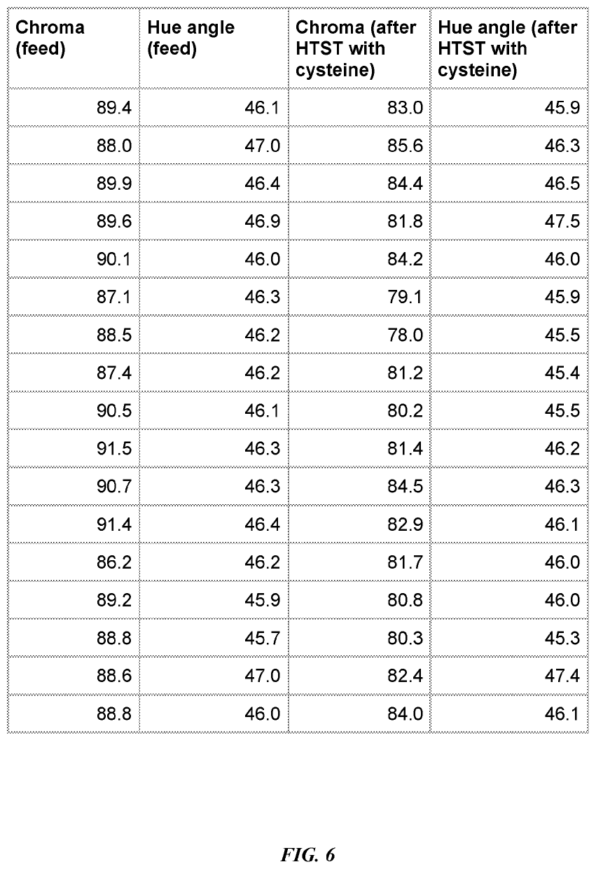

HTST treatment for 20 seconds at temperatures ranging from 68-72° C. resulted in a detrimental change in the colorimetric properties of LegH Prep (L*c*h). This color decline is due to LegH denaturation as well as potentially off-color formation produced by Pichia proteins in response to heat. In the presence of cysteine, the decline in L*c*h was diminished in a dose-dependent manner. These results support that cysteine may stabilize both LegH and Pichia proteins. FIG. 6 shows chroma and hue angle results before and after HTST with cysteine for a leghemoglobin solution. FIG. 6 was performed at 15 mM cysteine, a temperature of 65° C., and a duration of 20 seconds. Performing HTST in the presence of cysteine results in less change in both chroma and hue angle show a decrease after HTST compared to performing HTST in the absence of cysteine.

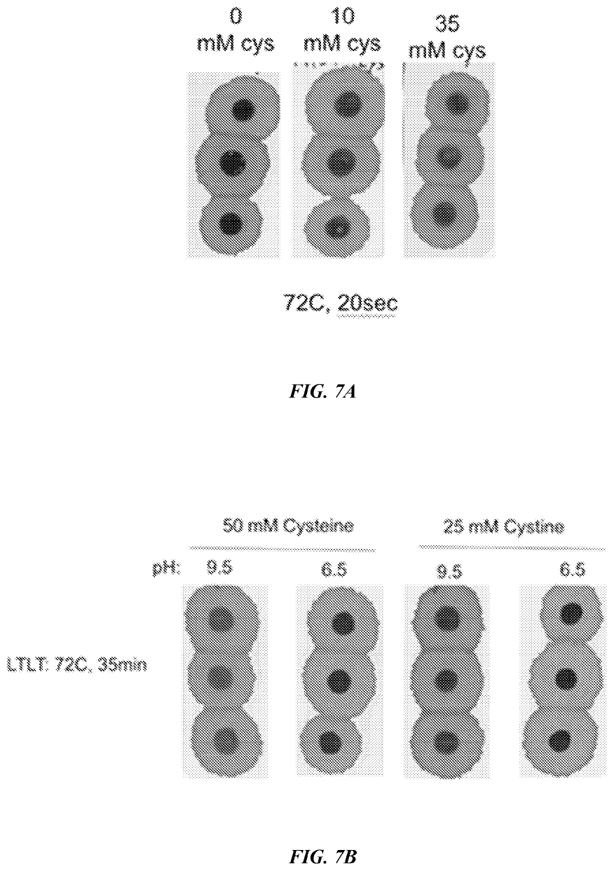

The off-color that forms when LegH Prep is exposed for 20 seconds at temperatures ranging from 68-72° C. is primarily localized to the suspended solids. Cysteine visually decreases this off-color formation (FIG. 7A).

Color protection of LegH Prep upon exposure to heat has cysteine in the reduced state (cysteine rather than cystine) and at high pH (FIG. 7B).

Other reductants such as sodium ascorbate may be used to reduce heme in its carrier proteins.

Thermal death time of pathogens in protein solutions is analyzed. Thermal processing conditions of protein solutions containing a heme-containing protein to eliminate Salmonella spp., L. monocytogenes, and E. coli O157 were studied. The study was conducted to develop D and z thermal death time (TDT) data for the selected pathogens. The strains are inoculated in the protein solutions, with or without cysteine, at pH 9.3. The TDT data generated can be used to validate that the process conditions achieve a 5-log reduction of these pathogens.

### A. Study Design

### 1. Inocula Preparation

The cultures used in these studies were the following:

Salmonella cocktail: Salmonella Senftenberg 775W (known to be heat resistant); Salmonella Senftenberg; Salmonella Montevideo FDA 488275 (known to be heat resistant); Salmonella FDA BAA-1045 (known to be heat resistant); Salmonella Agona FDA 447967 (known to be heat resistant).

Listeria monocytogenes strains: ATCC 19115; DSM 20600; CECT 5672; CECT 937; MEI 937.

E. coli strains: ATCC 35150; ATCC 43890; ATCC 43895; MEI 45403; MEI 35071.

Each culture was individually grown in a lawn by transferring an aliquot to tryptic soy agar with 0.6% yeast extract (TSAYE) plates and incubating at 35±2° C. for 18-24 h. Lawns were harvested by scraping the biomass off using sterile glass spreaders. Equal volumes of each culture strain were combined to make individual culture cocktails. The concentration of the inoculum was determined as described below.

### 2 Product and Product Inoculation

On the testing day, the frozen and aliquoted product was thawed. Cysteine and pH were adjusted. Product was inoculated and tested within 4 h after adjusting the pH. Fresh cysteine solution was made on each experiment day.

Samples: 100 mg/g LegH, pH 9.3, 30 mM Na-ascorbate, 15 mM cysteine were used.

### 3. Treatments

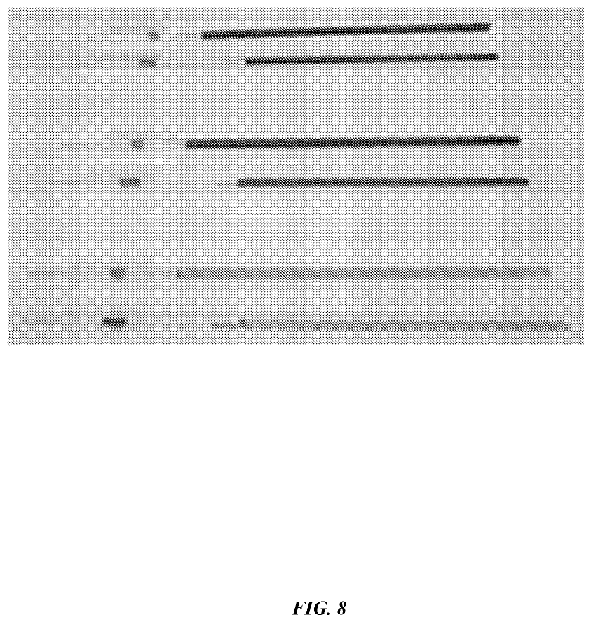

Capillary tubes (FIG. 8) were used to test the reduction of the testing organisms. These capillary tubes are usually employed in thermal death time experiments involving liquid products because the come-up time for the sample to achieve the testing temperature is short. FIG. 8 shows examples of product placed in 100 μl (blue) and 200 μl (red) capillary tubes. The first, third, and fifth tubes (when counting from top to bottom) are 200 μl (red) capillary tubes.

To prepare samples for thermal inactivation runs, 200 μL portions of inoculated product were injected into sterilized calibrated glass capillary tubes, leaving no air bubbles within the sample. Both ends of the capillary tube were sealed by melting the glass using a butane torch.

Temperature exposures were performed by dipping the capillary tubes into a temperature-controlled (+1° C.) hot water bath. After the treatment, the capillaries were quickly cooled by plunging them into room temperature water and dipping the tubes into 70% isopropanol. The surviving inoculum cells were enumerated by pulverizing the capillary tubes into Buffered Peptone Water (BPW) with tween 80 at a ratio of 1:10 and plating onto selective media as described below.

### 4. Enumeration of Target Organisms

Decimal serial dilutions were plated on selective media with a TSA overlay to recover injured cells. Salmonella were plated on XLD-TSA, E. coli O157 on SMAC-TSA, and Listeria on MOX-TSA agar plates. Plates were incubated at 35=2° C. for 18-24 h (Salmonella and E. coli) and 48 h (Listeria). Typical colonies were counted, and counts were transformed to Logarithm₁₀ for data analysis.

### 5. Estimation of the D and z Values

D values by temperature were estimated by averaging the final counts (CFU/g) and transforming to Logarithm₁₀ (Log) for each time-temperature condition. The resulting values were plotted, and linear regression analysis was applied to calculate the inverse slope as the D values (i.e., the time required at a given temperature to cause a 10-fold decrease in the microbial population).

The D values were then plotted against their respective temperatures and the inverse slope of the linear regression analysis yielded a z value (i.e., the change in temperature required to cause a tenfold change in D values).

### B. Results

| Test | | |
|---|---|---|
| run | Broth | Culture |
| 1 | 100 mg/g LegH, 15 mM Cysteine | Salmonella cocktail |
| 2 | 100 mg/g LegH, 15 mM Cysteine | L. monocytogenes cocktail |
| 3 | 100 mg/g LegH, 15 mM Cysteine | E. coli O157 cocktail |

### 1. Estimation of the D and z Values

The estimated D and z values of each broth were calculated after plotting the average counts at each temperature and are presented herein.

TABLE 1
D and z values of Salmonella spiked
in 100 mg/g LegH with 15 mM cysteine

| | | 100 mg/g LegH | | | | | |
|---|---|---|---|---|---|---|---|
| | | D-values (sec) | | | | | |
| | Cysteine | 56° | 58° | 60° | 62° | 64° | z |
| Organism(s) | concentration | C. | C. | C. | C. | C. | (° C.) |
| Salmonella | 15 mM | 29.0 | — | 13.8 | 4.9 | — | 5.15 |
| cocktail | Cysteine | | | | | | |

TABLE 2
D and z values of Listeria monocytogenes
spiked in 100 mg/g LegH with 15 mM cysteine

| | | 100 mg/g LegH | | | | | |
|---|---|---|---|---|---|---|---|
| | | D-values (sec) | | | | | |
| | Cysteine | 56° | 58° | 60° | 62° | 64° | z |
| Organism(s) | concentration | C. | C. | C. | C. | C. | (° C.) |
| L. monocytogenes | 15 mM | — | 28.2 | 11.4 | 4.9 | — | 5.26 |
| cocktail | Cysteine | | | | | | |

TABLE 3
D and z values of E. coli O157 spiked
in 100 mg/g LegH with 15 mM cysteine

| | | 100 mg/g LegH | | | | | |
|---|---|---|---|---|---|---|---|
| | | D-values (sec) | | | | | |
| | Cysteine | 56° | 58° | 60° | 62° | 64° | z |
| Organism(s) | concentration | C. | C. | C. | C. | C. | (° C.) |
| E. coli O157 | 15 mM | 22.7 | — | 4.0 | 2.4 | — | 5.98 |
| cocktail | Cysteine | | | | | | |

The average counts of each run and the thermal death time graphs are presented herein.

### 2. Five-Log Cycle Reduction Time Estimates

The time required to reduce the tested pathogens in 5 Log cycles is presented in Table 4.

TABLE 4
Time (sec) required to reduce 5 log cycles of Salmonella, E. coli
O157, and L. monocytogenes in 100 m/g LegH with 15 mM cysteine

| | 100 mg/g LegH and 15 mM cysteine | | | | | |
|---|---|---|---|---|---|---|
| | 5 Log reduction time estimates (s) | | | | | |
| | 60° | 61° | 62° | 63° | 64° | 65° |
| Organism | C. | C. | C. | C. | C. | C. |
| Salmonella cocktail | 63 | 40 | 26 | 17 | 11 | 7 |
| E. coli O157 cocktail | 24 | 16 | 11 | 8 | 5 | 4 |
| L. monocytogenes | 59 | 38 | 25 | 16 | 11 | 7 |
| cocktail | | | | | | |

### C. Additional Results

1. Average Salmonella Cocktail Concentration in 100 mg/g LegH with 15 mM Cysteine at pH 9.3

| Treatment | Time-point | Salmonella (Log |
|---|---|---|
| temperature | (s) | CFU/mL ± SD) |
| 58° C. | 0 | 8.2 ± 0.0^{a} |
| (136.4° F.) | 20 | 6.4 ± 0.3 |
| | 80 | 4.4 ± 0.0 |
| | 130 | 3.4 ± 0.7 |
| 60° C. | 0 | 6.7 ± 0.2 |
| (140.0° F.) | 25 | 4.2 ± 0.1 |
| | 50 | 3.4 ± 0.3 |
| | 55 | 2.3 ± 0.1 |
| 62° C. | 0 | 6.9 ± 0.3 |
| (143.6° F.) | 9 | 4.0 ± 0.2 |
| | 18 | 3.1 ± 0.5 |
| | 23 | 1.9 ± 0.6 |

Log CFU/mL ± standard deviation

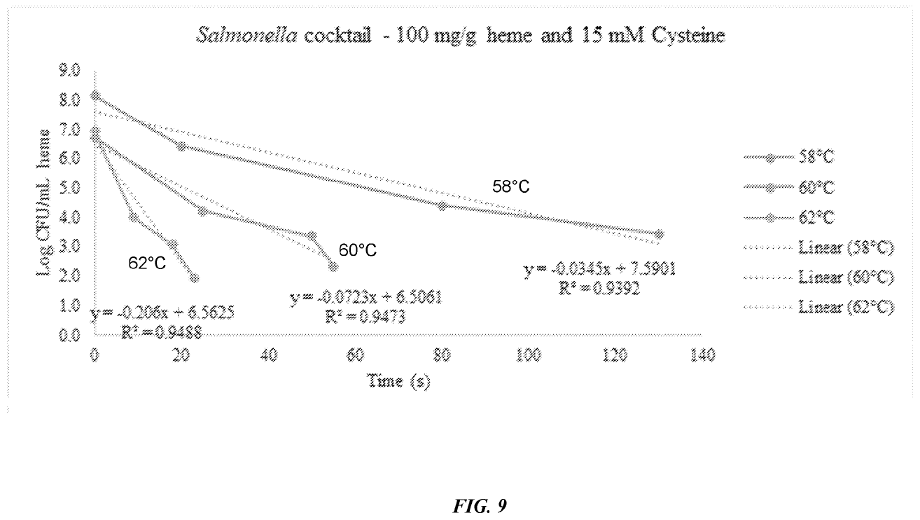

FIG. 9 shows the thermal inactivation of Salmonella cocktail at 58, 60, and 62° C. in 100 mg/g LegH and 15 mM cysteine.

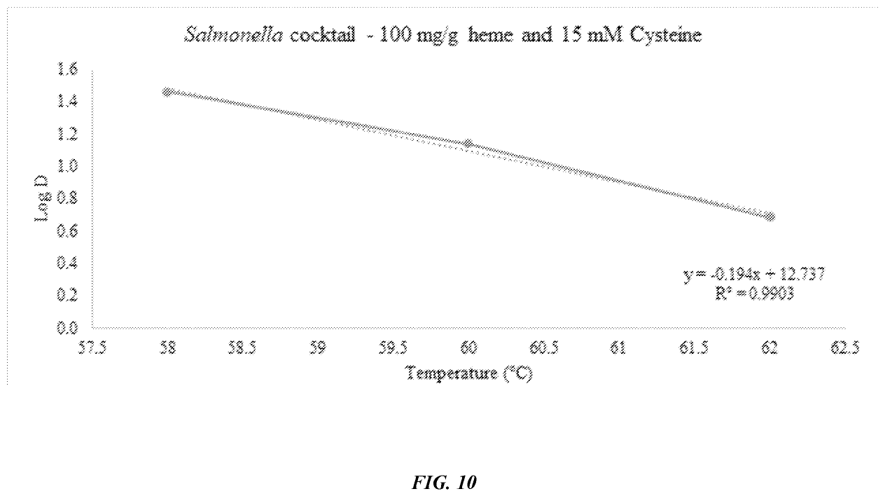

FIG. 10 shows D value estimates vs. temperature for Salmonella cocktail at 58, 60, and 62° C. in 100 mg/g LegH and 15 mM cysteine.

2. Average Counts L. monocytogenes Cocktail Spiked in 100 mg/g LegH with 15 mM Cysteine

Average Listeria monocytogenes cocktail concentration in 100 mg/g LegH with 15 mM Cysteine at pH 9.3

| Treatment | Time-point | L. monocytogenes |
|---|---|---|
| temperature | (s) | (Log CFU/mL ± SD) |
| 58° C. | 0 | 7.7 ± 0.1^{a} |
| (136.4° F.) | 60 | 5.5 ± 0.4 |
| | 100 | 4.0 ± 0.1 |
| | 140 | 2.8 ± 0.5 |
| 60° C. | 0 | 7.9 ± 0.1 |
| (140.0° F.) | 30 | 5.4 ± 0.1 |
| | 45 | 4.3 ± 0.1 |
| | 60 | 2.5 ± 0.2 |
| 62° C. | 0 | 7.9 ± 0.1 |
| (143.6° F.) | 10 | 4.8 ± 0.1 |
| | 20 | 3.6 ± 0.0 |
| | 30 | 1.5 ± 0.2 |

Log CFU/mL ± standard deviation

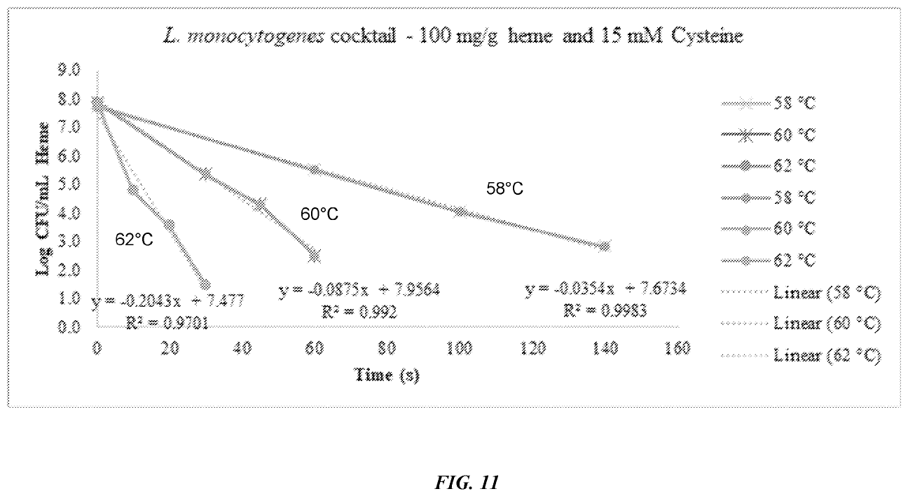

FIG. 11 shows thermal inactivation of L. monocytogenes cocktail at 58, 60, and 62° C. in 100 mg/g LegH and 15 mM cysteine.

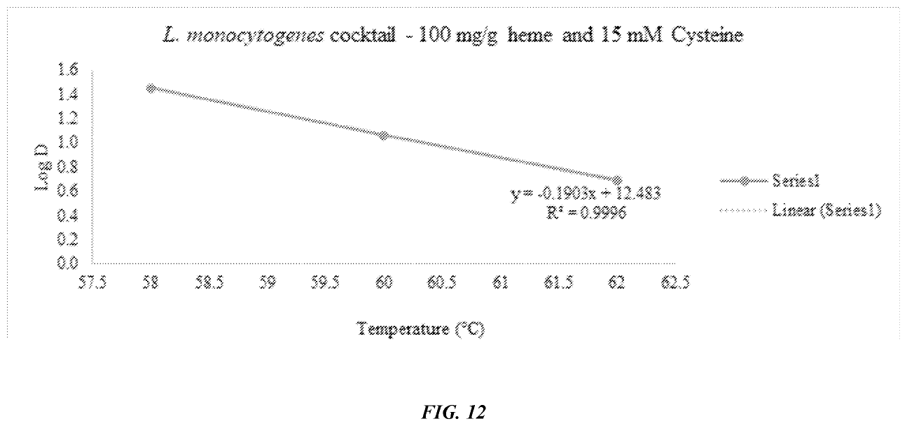

FIG. 12 shows D value estimates vs. temperature for L. monocytogenes cocktail at 58, 60, and 62° C. in 100 mg/g LegH and 15 mM cysteine.

3. Average Counts E. coli O157 Cocktail Spiked in 100 mg/g LegH with 15 mM Cysteine

Average E. coli O157 cocktail concentration in 100 mg/g LegH with 15 mM Cysteine at pH 9.3

| | | E. coli O157 |
|---|---|---|
| Treatment | Time-point | cocktail (Log |
| temperature | (s) | CFU/mL ± SD) |
| 56° C. | 0 | 8.2 ± 0.1^{a} |
| (132.8° F.) | 40 | 5.9 ± 0.3 |
| | 70 | 4.5 ± 0.4 |
| | 100 | 3.9 ± 0.5 |
| 60° C. | 0 | 8.1 ± 0.1 |
| (140.0° F.) | 8 | 6.5 ± 0.1 |
| | 12 | 5.3 ± 0.3 |
| | 20 | 3.1 ± 0.1 |
| 62° C. | 0 | 7.7 ± 0.1 |
| (143.6° F.) | 3 | 6.8 ± 0.2 |
| | 9 | 3.6 ± 0.2 |
| | 12 | 1.6 ± 0.2 |

Log CFU/mL ± standard deviation

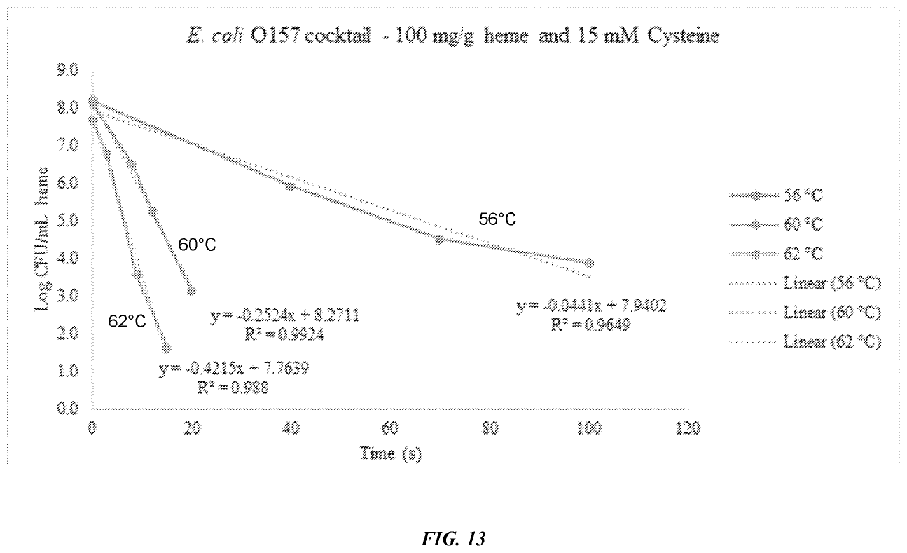

FIG. 13 shows thermal inactivation of E. coli O157 cocktail at 56, 60, and 62° C. in 100 mg/g LegH and 15 mM cysteine.

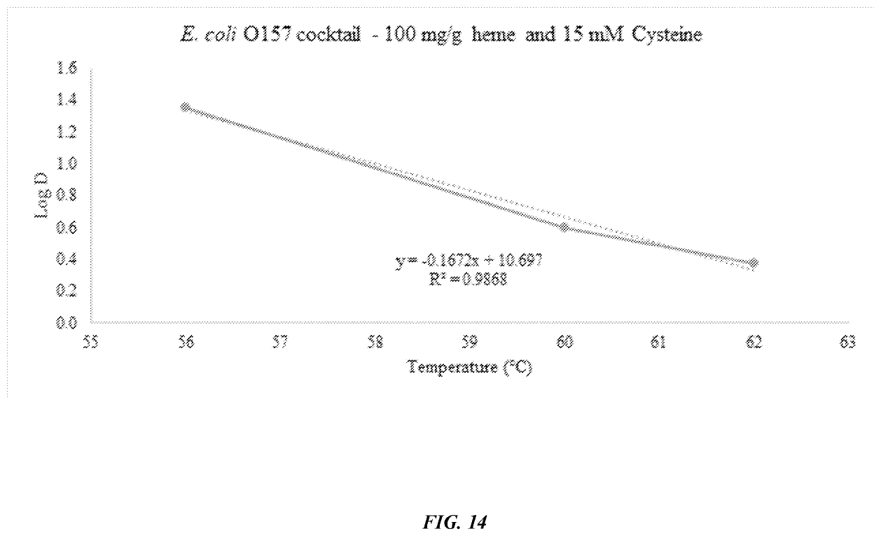

FIG. 14 shows D value estimates vs. temperature for E. coli O157 cocktail at 56, 60, and 62° C. in 100 mg/g LegH and 15 mM cysteine.

### III. Example Methods

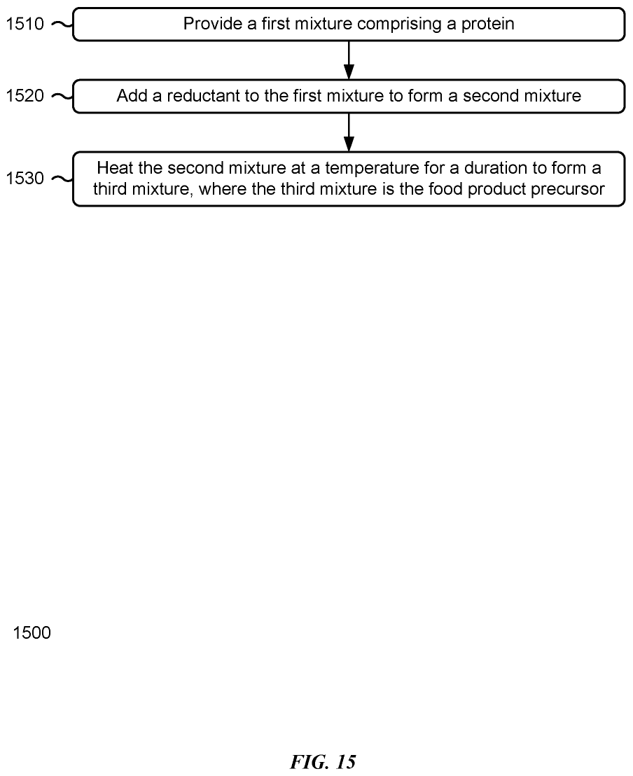

FIG. 15 is a flowchart of an example process 1500 associated forming a food product precursor. In some implementations, one or more process blocks of FIG. 15 may be performed by a device or system.

At block 1510, process 1500 may include providing a first mixture comprising a protein. The first mixture may be a solution or suspension. The first mixture may have a total protein concentration of 10 mg/g or higher, including 10 to 20, 20 to 30, 30 to 40, 40 to 50, 50 to 60, 60 to 70, 70 to 80, 80 to 90, 90 to 100, or 100 to 200, or greater than 200 mg/g. The protein may be a metalloprotein. The metalloprotein may have iron, zinc, manganese, cobalt, copper, calcium, vanadium, magnesium, cadmium, molybdenum, or tungsten as the metal ion cofactor. The metalloprotein may be an iron-containing protein. The iron-containing protein may be a heme-containing protein. The heme-containing protein may be leghemoglobin or myoglobin. The protein may be recombinantly produced.

The heme-containing protein may be a globin. The globin may be PF00042 in the Pfam database. The globin may be a cytochrome (e.g., a cytochrome P450, a cytochrome a, a cytochrome b, a cytochrome c), a cytochrome c oxidase, a ligninase, a catalase, or a peroxidase. In addition, the globin may be an androglobin, a chlorocruorin, a cytoglobin, an erythrocruorin, a flavohemoglobin, a globin E, a globin X, a globin Y, a hemoglobin (e.g., a beta hemoglobin, an alpha hemoglobin), a histoglobin, a leghemoglobin, a myoglobin, a neuroglobin, a non-symbiotic hemoglobin, a protoglobin, or a truncated hemoglobin (e.g., a HbN, a HbO, a Glb3, a cyanoglobin).

In some embodiments, the protein may be an enzyme. The enzyme may include a metalloenzyme, where iron, zinc, manganese, cobalt, copper, calcium, vanadium, magnesium, cadmium, molybdenum, or tungsten may be the metal ion cofactor. The enzyme may be a dehydrin, phytase, protease, catalase, lipase, peroxidase, amylase, transglutaminase, oxidoreductase, transferase, hydrolase, lyase, isomerase, ligase, amylase, mannanase, licheninase, or cellulase.

The protein may be a redox active protein, an oxygen binding or oxygen carrying protein, an electron transfer protein, an iron-sulfur protein, or a ferredoxin protein. The protein may include a biologic, an antibody, an antibody fragment, an antibody-drug conjugate, an antigen, a regulatory protein, a peptide hormone, a blood clotting protein, a cytokine, or a cytokine inhibitor. The protein may be a cysteine-containing protein, a protein with an exposed surface thiol group, a protein that can form an intramolecular or intermolecular disulfide bond, or a protein that can participate in thiol-disulfide exchange. The protein may be a cytosolic protein, a seed storage protein, ribulose-1,5-bisphosphate carboxylase/oxygenase (Rubisco), ovalbumin, or lactalbumin. The protein may be a protein with a denaturation temperature, aggregation temperature, or enzyme inactivation temperature of above 80° C., above 75° C., above 70° C., above 65° C., or above 60° C., or a temperature in a range between any two of these temperatures. The protein may be a protein with color, including chromoprotein, pigment-protein complex, phycobiliprotein, phycocyanin, phytochrome, hemerythrin, chlorocruorin, vanabin, erythrocruorin, pinnaglobin, coboglobin, or hemocyanin. Methods may include one or more of the proteins described herein.

At block 1520, process 1500 may include adding a reductant to the first mixture to form a second mixture. The protein in the second mixture may have a concentration in a range of 10 mg/g to 200 mg/g. The concentration may be 10 to 20, 20 to 30, 30 to 40, 40 to 50, 50 to 60, 60 to 70, 70 to 80, 80 to 90, 90 to 100, or 100 to 200, or greater than 200 mg/g. The reductant may be cysteine. The reductant may be food safe. For example, the reductant may be categorized by the U.S. Food and Drug Administration as GRAS (generally recognized as safe). The reductant may appear in the Select Committee on GRAS Substances (SCOGS) database (available at www.cfsanappsexternal.fda.gov/scripts/fdcc/?set=SCOGS), the contents of which are incorporated herein by reference. The reductant may be glutathione. The reductant may be bisulfite, sodium metabisulfite, or hydrogen sulfite. The reductant may include a sulfite. The reductant may include a nitrogen-containing compound (e.g., ammonia).

The reductant in the second mixture may have a concentration in a range from 1 mM to 150 mM, 1 mM to 2.5 mM, 2.5 mM to 5 mM, 5 mM to 50 mM, 50 mM to 100 mM, 100 mM to 125 mM, or 125 mM to 150 mM.

The second mixture may further include a pH buffer. The second mixture may have a pH of 8 or higher, a pH of 9 or higher, a pH of 10 or higher. The second mixture may have a pH in a range from 8 to 9, 9 to 10, 10 to 10.5, 10.5 to 11, 11 to 12, 12 to 12.5, 12.5 to 13, or greater than 13.

In embodiments, the method further comprises adding a second reductant to the first mixture. The second reductant may be sodium ascorbate or any reductant described herein.

At block 1530, process 1500 may include heating the second mixture at a temperature for a duration to form a third mixture, where the third mixture is the food product precursor. The third mixture may be any food product precursor described herein.

The third mixture may have a chroma in a range from 78 to 86, 60 to 70, 70 to 78, 78 to 80, 80 to 86, or 86 to 90. The change in chroma between the first mixture and the third mixture may be in a range from 0 to 20, 0 to 10, 2 to 11, 0 to 5, 5 to 10, 10 to 15, or 15 to 20. The chroma in the third mixture may be lower than the first mixture. The third mixture may have a hue angle in a range from 45 to 48, 40 to 45, 48 to 50, or 50 to 55. The change in hue angle between the first mixture and the third mixture may be in a range from 0 to 10, 0 to 20, 0 to 5, 0.5 to 0.9, 5 to 10, 10 to 15, or 15 to 20. The hue angle in the third mixture may be lower than the first mixture.

The temperature may be 80° C. or less. The temperature may be in a range from 60 to 75° C., 60 to 65° C., 65 to 70° C., 70 to 75° C., or 75 to 80° C.

The duration may be in a range from 1 second to 10 minutes, including 1 second to 30 seconds, 30 seconds to 1 minute, 1 to 2 minutes, 2 to 5 minutes, or 5 to 10 minutes. The duration may be 30 seconds or less.

In embodiments, 25% or less of the protein in the third mixture is denatured compared to the protein in the second mixture, including 20% to 25%, 15% to 20%, 10% to 15%, 5% to 10%, 1% to 5%, or 0% to 1%.

In embodiments, the food product precursor further comprises a preservative. Process 1500 may include adding the preservative to the first mixture, the second mixture, or the third mixture.

The food product precursor may have an aerobic plate count of 100,000 colony forming units per gram or less, 50,000 colony forming units per gram or less, 20,000 colony forming units per gram or less, 10,000 colony forming units per gram or less, 5,000 colony forming units per gram or less, 1,000 colony forming units per gram or less, 500 colony forming units per gram or less, or 100 colony forming units per gram or less.

After heating the second mixture, the heating may be stopped. The third mixture may be diluted. The dilution may occur before heating. In some embodiments, the third mixture may be packaged in an airtight container.

Process 1500 may include additional implementations, such as any single implementation or any combination of implementations described below and/or in connection with one or more other processes described elsewhere herein.

In embodiments, the protein may be a first protein. The first mixture may include a second protein. The second protein may be a protein from the host cell when the first protein is recombinantly produced. The second protein may be a microbial protein. The second protein may be a fungal, bacterial, or algal protein. The second protein may be a Pichia protein. The second protein may be a cytosolic protein. The second protein may be any protein described herein.

Process 1500 may include lysing a plurality of cells to obtain a cell lysate. In embodiments, the cell lysate may be the first mixture. Lysing may be mechanical, chemical, or biochemical. For example, mechanical lysing may include sonication, bead milling, osmotic lysis, homogenization, manual grinding, or subjecting the cells to freeze-thaw cycles. Chemical lysing may include surfactant-based lysis, chaotropic-based lysis, or organic solvent-based lysis. Biochemical lysis may include enzymatic cell wall degradation.

In some embodiments, process 1500 may further include clarifying the cell lysate to obtain a clarified lysate. In embodiments, process 1500 may further include filtering the clarified lysate to obtain the first mixture. The cells may be fungal, bacterial, or algal cells.

Process 1500 may include lysing a plurality of cells in the second mixture during heating. The cells may be cells of one or more foodborne pathogens or food spoilage microbes. Process 1500 may include aggregating insoluble solids in the third mixture. The insoluble solids may not include the protein Process 1500 may include removing the insoluble solids from the third mixture. In some embodiments, process 1500 may prevent or reduce the formation of insoluble solids.

The second mixture may have a first protein concentration of the protein. The third mixture may have a second protein concentration of the protein. The second protein concentration may be less than the first protein concentration. A first difference between the first protein concentration and the second protein concentration may be less than a second difference between concentrations before and after heating without adding the reductant.

The second mixture may have a first suspended solids percentage. The third mixture may have a second suspended solids percentage. The second suspended solids percentage may be greater than the first suspended solid percentage. A first difference between the first suspended solids percentage and the second suspended solids percentage may be less than a second difference between percentages before and after heating without adding the reductant. The first suspended solids percentage may be 5% or less, including 0% to 1%, 1% to 2%, 2% to 3%, 3% to 4%, or 4% to 5%. The second suspended solids percentage may be 60% or less, including 0% to 10%, 10% to 20%, 20% to 30%, 30% to 40%, 40% to 50%, or 50% to 60%. The first difference may be 30% or less, including 0% to 10%, 10% to 15%, 15% to 20%, 20% to 25%, or 25% to 30%. The second difference may be 10% or higher, including 10% to 15%, 15% to 20%, 20% to 25%, 25% to 30%, or 30% or higher. In some embodiments, the suspended solids are insoluble solids.

The second mixture may have a first microbial concentration of one or more microbes. The third mixture may have a second microbial concentration of the one or more microbes. The second microbial concentration may be less than the first microbial concentration. A first difference between the first microbial concentration and the second microbial concentration may be greater than a second difference between concentrations before and after heating without adding the reductant. The one or more microbes may include one or more foodborne pathogens or food spoilage microbes. The one or more microbes may include eukaryotes, prograyotes, or archae bacteria. The one or more microbes may be selected from Salmonella, Listeria monocytogenes, and E. coli. The one or more microbes may include Gram-positive bacteria, Gram-negative bacteria, a mold, a yeast, an algae, or a parasite. The Gram-positive bacteria may be selected from Staphylococcus aureus, Bacillus spp, Clostridium spp, Lactic acid bacteria, Carnobacterium spp., Lactobacillus spp., Leuconostoc spp., Streptococcus spp., Lactococcus spp., Brochothrix spp., Weissella spp., Pediococcus spp., Kurthia zopfii, and Mycobacterium bovis. The Gram-negative bacteria may be selected from Salmonella spp., Shigella, Vibrio spp., Escherichia coli, Campylobacter jejuni, Yersinia enterocolitis, Brucella spp., Coxiella burnetii, Aeromonas spp., and Plesiomonas shigelloides.

The mold may be selected from Mucor, Aspergillus spp., Rhizopus spp., Penicillium spp., Alternaria spp., Bothrytis, Byssochlamys, and Fusarium spp.

The yeast may be selected from Zygosaccharomyces spp, Saccharomyces spp., Pichia spp., Candida spp., and Dekkera spp.

The parasite may be selected from Giardia lamblia, Entamoeba histolytica, Cyclospora cayetanensis, Toxoplasma gondii, and Trichinella spiralis.

The algae may be selected from Gonyaulax catenella, Gonyaulax tamarensis, Gambierdiscus toxicus, Ptychodiscus brevis, Microcystis aeruginosa, and blue-green algae.

The first difference may be at least a 2-log, 3-log, 4-log, or 5-log reduction. The reduction may be of at least one microbe, at least two microbes, at least three microbes, or at least four microbes.

The second microbial concentration is an aerobic plate count of 100,000 colony forming units per gram or less, 50,000 colony forming units per gram or less, 20,000 colony forming units per gram or less, 10,000 colony forming units per gram or less, 5,000 colony forming units per gram or less, 1,000 colony forming units per gram or less, 500 colony forming units per gram or less, or 100 colony forming units per gram or less.

Although FIG. 15 shows example blocks of process 1500, in some implementations, process 1500 may include additional blocks, fewer blocks, different blocks, or differently arranged blocks than those depicted in FIG. 15. Additionally, or alternatively, two or more of the blocks of process 1500 may be performed in parallel. Methods may involve methods disclosed in U.S. Pat. No. 11,051,532 or WO 2022/055513 A1 filed Sep. 14, 2020, the contents of both of which are incorporated herein in its entirety for all purposes.

Embodiments may include a method of reducing bioburden in a first mixture. The method may be performed in a similar manner as process 1500. The method may include providing the first mixture, which may be performed similar to block 1510. The first mixture may include the protein. The first mixture may be an aqueous composition. The method may include adding a reductant to the first mixture to form a second mixture, which may be performed similar to block 1520. The method may include heating the second mixture at a temperature for a duration to form a third mixture, which may be performed in a similar manner as block 1530. The third mixture may be the food product precursor. The third mixture has a lower bioburden than the first mixture.

The first mixture may be a protein solution. The protein may be a protein for pharmaceuticals or other proteins. The third mixture may not be a food product precursor. The reductant may not be food safe. The reductant may include dithiothreitol (DTT), tris (2-carboxyethyl) phosphine) (TCEP), or sodium dithionate.

### IV. Example Compositions

Embodiments may include a food product precursor. The food product precursor may include a heme-containing protein. The food product precursor may include a reductant having a first concentration in a range of 5 mM to 50 mM, including 5 mM to 10 mM, 10 mM to 20 mM, 20 mM to 30 mM, 30 mM to 40 mM, or 40 mM to 50 mM. The reductant concentration may be the concentration in oxidized form. The reductant may be any reductant described herein. The food product precursor may have a pH of 5.5 or higher. The pH may be in a range from 5.5 to 6, 6 to 7, 7 to 8, 8 to 8.5, 8.5 to 9, or greater than 9. The pH may be any pH described herein. The food product precursor may be a solution or suspension. The food product precursor may be formed by process 1500 or any method described herein.

The heme-containing protein may be leghemoglobin or myoglobin. The heme-containing protein may have a second concentration in the range of 10 mg/g to 200 mg/g. The heme-containing protein may have a second concentration that is equal to any protein concentration described herein. The heme-containing protein may include 50% or higher of total protein content in the food product precursor, including 50% to 60%, 60% to 70%, 70% to 80%, 80% to 90%, 90% to 95%, 95% to 99%, or 99% to 100%. The heme-containing protein may have greater than 50% iron in Fe (II) reduced state, including 50% to 60%, 60% to 70%, 70% to 80%, 80% to 90%, 90% to 95%, 95% to 99%, or 99% to 100%.

The food product precursor may have an aerobic plate count of 100,000 colony forming units per gram or less, 50,000 colony forming units per gram or less, 20,000 colony forming units per gram or less, 10,000 colony forming units per gram or less, 5,000 colony forming units per gram or less, 1,000 colony forming units per gram or less, 500 colony forming units per gram or less, or 100 colony forming units per gram or less.

The food product precursor may have a refrigerated shelf life over 30 days.

The food product precursor may include a fungal, bacterial, or algal cytosolic protein. The food product precursor may include a Pichia protein, sodium ascorbate, or a preservative.

The food product precursor may have a suspended solids percentage less than 60%, including 0% to 10%, 10% to 20%, 20% to 30%, 30% to 40%, 40% to 50%, or 50% to 60%.

The food product precursor may have a hue angle in a range from 34 to 40 degrees, 40 to 45 degrees, 45 to 48 degrees, or 48 to 50 degrees, or any hue angle described herein.

The food product precursor may have a chroma over 21, including in a range from 78 to 86, 86 to 92, 70 to 78, or any chroma described herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure. Percentages herein may be by mass, volume, mole, or parts.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

1. A method for forming a food product precursor, the method comprising:
providing a first mixture comprising a protein;
adding a reductant to the first mixture to form a second mixture;
heating the second mixture at a temperature for a duration to form a third mixture, wherein the third mixture is the food product precursor.

2. A method of reducing bioburden in a first mixture, the method comprising:
providing the first mixture comprising a protein;
adding a reductant to the first mixture to form a second mixture; and
heating the second mixture at a temperature for a duration to form a third mixture, wherein the third mixture has a lower bioburden than the first mixture.

3. The method of claim 1 or 2, wherein the first mixture is a solution or suspension.

4. The method of claim 1 or 2, wherein the first mixture has a total protein concentration of 10 mg/g or higher.

5. The method of claim 1 or 2, wherein the protein is a metalloprotein.

6. The method of claim 5, wherein the metalloprotein is an iron-containing protein.

7. The method of claim 6, wherein the iron-containing protein is a heme-containing protein.

8. The method of claim 7, wherein the heme-containing protein is leghemoglobin or myoglobin.

9. The method of claim 7, wherein the third mixture has a chroma in a range from 78 to 86.

10. The method of claim 7, wherein the third mixture has a hue angle in a range from 45 to 48.

11. The method of claim 7, wherein the change in chroma between the first mixture and the third mixture is in a range from 2 to 11.

12. The method of claim 7, wherein the change in hue angle between the first mixture and the third mixture is in a range from 0.5 to 0.9.

13. The method of claim 1 or 2, wherein the protein is recombinantly produced.

14. The method of claim 1 or 2, wherein the protein in the second mixture has a concentration in a range of 10 mg/g to 200 mg/g.

15. The method of claim 1 or 2, wherein the reductant is a sulfur-containing compound.

16. The method of claim 1 or 2, wherein the reductant is cysteine.

17. The method of claim 1 or 2, wherein the reductant is glutathione.

18. The method of claim 1 or 2, wherein the reductant is bisulfite.

19. The method of claim 1 or 2, wherein the reductant is food safe.

20. The method of claim 1 or 2, wherein the reductant in the second mixture has a concentration in a range from 1 mM to 150 mM.

21. The method of claim 1 or 2, wherein the reductant in the second mixture has a concentration in a range from 5 mM to 50 mM.

22. The method of claim 1 or 2, wherein the second mixture further comprises a pH buffer.

23. The method of claim 1 or 2, wherein the second mixture has a pH of 9 or higher.

24. The method of claim 1 or 2, wherein the second mixture has a pH of 10 or higher.

25. The method of claim 1 or 2, wherein the second mixture has a pH in a range from 9 to 10.

26. The method of claim 1 or 2, wherein the second mixture has a pH in a range from 10 to 10.5.

27. The method of claim 1 or 2, wherein the temperature is 80° C. or less.

28. The method of claim 1 or 2, wherein the temperature is in a range from 60 to 75° C.

29. The method of claim 1 or 2, wherein the duration is in a range from 1 second to 10 minutes.

30. The method of claim 1 or 2, wherein the duration is 30 seconds or less.

31. The method of claim 1 or 2, wherein 25% or less of the protein in the third mixture is denatured compared to the protein in the second mixture.

32. The method of claim 1, wherein the food product precursor further comprises a preservative.

33. The method of claim 1, wherein the food product precursor has an aerobic plate count of 100,000 colony forming units per gram or less.

34. The method of claim 1, wherein the food product precursor has an aerobic plate count of 10,000 colony forming units per gram or less.

35. The method of claim 1 or 2, wherein:
the protein is a first protein,
the first mixture comprises a second protein, and
the second protein is a Pichia protein.

36. The method of claim 1 or 2, wherein:
the reductant is a first reductant, and
the method further comprises adding a second reductant to the first mixture.

37. The method of claim 36, wherein the second reductant is sodium ascorbate.

38. The method of claim 1 or 2, further comprising:
lysing a plurality of cells to obtain a cell lysate,
clarifying the cell lysate to obtain a clarified lysate, and
filtering the clarified lysate to obtain the first mixture.

39. The method of claim 38, wherein the cells are fungal, bacterial, or algal cells.

40. The method of claim 1 or 2, further comprising:
lysing a plurality of cells in the second mixture during heating, wherein the cells are cells of one or more foodborne pathogens or food spoilage microbes.

41. The method of claim 1 or 2, further comprising:
aggregating insoluble solids in the third mixture, wherein the insoluble solids do not include the protein, and
removing the insoluble solids from the third mixture.

42. The method of claim 1 or 2, wherein:
the second mixture comprises a first protein concentration of the protein,
the third mixture comprises a second protein concentration of the protein,
the second protein concentration is less than the first protein concentration, and
a first difference between the first protein concentration and the second protein concentration is less than a second difference between concentrations before and after heating without adding the reductant.

43. The method of claim 1 or 2, wherein:
the second mixture comprises a first suspended solids percentage,
the third mixture comprises a second suspended solids percentage,
the second suspended solids percentage is greater than the first suspended solid percentage, and
a first difference between the first suspended solids percentage and the second suspended solids percentage is less than a second difference between percentages before and after heating without adding the reductant.

44. The method of claim 43, wherein the first suspended solids percentage is 5% or less.

45. The method of claim 43, wherein the second suspended solids percentage is 60% or less.

46. The method of claim 43, wherein the second difference is 30% or higher.

47. The method of claim 1 or 2, wherein:
the second mixture comprises a first microbial concentration of one or more microbes,
the third mixture comprises a second microbial concentration of the one or more microbes,
the second microbial concentration is less than the first microbial concentration, and
a first difference between the first microbial concentration and the second microbial concentration is greater than a second difference between concentrations before and after heating without adding the reductant.

48. The method of claim 47, wherein the one or more microbes comprise one or more foodborne pathogens or food spoilage microbes.

49. The method of claim 47, wherein the one or more microbes are selected from Salmonella, Listeria monocytogenes, and E. coli.

50. The method of claim 47, wherein the one or more microbes comprise Gram-positive bacteria, Gram-negative bacteria, a mold, a yeast, an algae, or a parasite.

51. The method of claim 50, wherein the Gram-positive bacteria is selected from Staphylococcus aureus, Bacillus spp, Clostridium spp, Lactic acid bacteria, 2 Carnobacterium spp., Lactobacillus spp., Leuconostoc spp., Streptococcus spp., Lactococcus spp., Brochothrix spp., Weissella spp., Pediococcus spp., Kurthia zopfii, and Mycobacterium bovis.

52. The method of claim 50, wherein the Gram-negative bacteria is selected from Salmonella spp., Shigella, Vibrio spp., Escherichia coli, Campylobacter jejuni, Yersinia enterocolitis, Brucella spp., Coxiella burnetii, Aeromonas spp., and Plesiomonas shigelloides.

53. The method of claim 50, wherein the molds is selected from Mucor, Aspergillus spp., Rhizopus spp., Penicillium spp., Alternaria spp., Bothrytis, Byssochlamys, and Fusarium spp.

54. The method of claim 50, wherein the yeast is selected from Zygosaccharomyces spp, Saccharomyces spp., Pichia spp., Candida spp., and Dekkera spp.

55. The method of claim 50, wherein the parasite is selected from Giardia lamblia, Entamoeba histolytica, Cyclospora cayetanensis, Toxoplasma gondii, and Trichinella spiralis.

56. The method of claim 50, wherein the algae is selected from Gonyaulax catenella, Gonyaulax tamarensis, Gambierdiscus toxicus, Ptychodiscus brevis, Microcystis aeruginosa, and blue-green algae.

57. The method of claim 47, wherein the first difference is at least a 2-log reduction.

58. The method of claim 47, wherein the first difference is at least a 5-log reduction.

59. The method of claim 47, wherein the second microbial concentration is an aerobic plate count of 100,000 colony forming units per gram or less.

60. The method of claim 47, wherein the second microbial concentration is an aerobic plate count of 10,000 colony forming units per gram or less.

61. The method of claim 1 or 2, wherein the change in chroma between the first mixture and the third mixture is in a range from 0 to 20.

62. The method of claim 1 or 2, wherein the change in chroma between the first mixture and the third mixture is in a range from 0 to 10.

63. The method of claim 1 or 2, wherein the change in hue angle between the first mixture and the third mixture is in a range from 0 to 20.

64. The method of claim 1 or 2, wherein the change in hue angle between the first mixture and the third mixture is in a range from 0 to 10.

65. A food product precursor, the food product precursor comprising:
a heme-containing protein; and
cysteine having a first concentration in a range of 5 mM to 50 mM;
wherein the food product precursor has a pH of 5.5 or higher.

66. The food product precursor of claim 65, wherein the heme-containing protein is leghemoglobin or myoglobin.

67. The food product precursor of claim 66, wherein the heme-containing protein has a second concentration in the range of 10 mg/g to 200 mg/g.

68. The food product precursor of claim 65, wherein the heme-containing protein comprises 50% or higher of total protein content in the food product precursor.

69. The food product precursor of claim 65, wherein the food product precursor is a solution or suspension.

70. The food product precursor of claim 65, wherein the food product precursor has an aerobic plate count of 100,000 colony forming units per gram or less.

71. The food product precursor of claim 65, wherein the food product precursor has an aerobic plate count of 10,000 colony forming units per gram or less.

72. The food product precursor of claim 65, wherein the food product precursor has a refrigerated shelf life over 30 days.

73. The food product precursor of claim 65, further comprising a fungal, bacterial, or algal cytosolic protein.

74. The food product precursor of claim 65, further comprising a Pichia protein.

75. The food product precursor of claim 65, further comprising sodium ascorbate.

76. The food product precursor of claim 65, further comprising a preservative.

77. The food product precursor of claim 65, wherein the food product precursor has a suspended solids percentage less than 60%.

78. The food product precursor of claim 65, wherein the food product precursor has a chroma in a range from 86 to 92.

79. The food product precursor of claim 65, wherein the food product precursor has a hue angle from 45 to 48.

80. The food product precursor of claim 65, wherein the food product precursor has a chroma in a range from 78 to 86.
